(19) United States
(12) Patent Application Publication
(10) Pub. No.: US 2016/0361334 A1
(43) Pub. Date: Dec. 15, 2016

(54) APPLICATION OF BAICALIN IN PREPARATION OF DRUG FOR TREATING RICIN POISONING

(71) Applicants: JILIN UNIVERSITY, Jilin (CN); INSTITUTE OF BIOPHYSICS, CHINESE ACADEMY OF SCIENCES, Beijing (CN)

(72) Inventors: Xuming DENG, Jilin (CN); Zihe RAO, Jilin (CN); Jing DONG, Jilin (CN); Xuemei LI, Jilin (CN); Yong ZHANG, Jilin (CN); Yutao CHEN, Jilin (CN); Jiazhang QIU, Jilin (CN)

(73) Assignees: JILIN UNIVERSITY, Jilin (CN); INSTITUTE OF BIOPHYSICS, CHINESE ACADEMY OF SCIENCES, Beijing (CN)

(21) Appl. No.: 15/116,793

(22) PCT Filed: Apr. 29, 2014
(86) PCT No.: PCT/CN2014/076439

§ 371 Date: Aug. 4, 2016

(30) Foreign Application Priority Data
Feb. 26, 2014 (CN) 201410066491.X

(51) Int. Cl. A61K 31/7048

(52) U.S. Cl. CPC A61K 31/7048

(57) ABSTRACT

Provided is an application of baicalin in preparation of a drug for treating ricin poisoning. A Hela cytoprotection test and a mouse ricin poisoning model have verified the recovery effect of baicalin on ricin poisoning.

### FIELD OF THE INVENTION

The present invention belongs to the field of medicine, and in particular relates to a use of baicalin in the preparation of a drug for treating ricin poisoning.

### BACKGROUND OF THE INVENTION

Baicalin is a flavonoid extracted and isolated from some plants, such as Chinese herbs Scutellaria baicalensis Georgi and Oroxylum indicum, and has significant antifungal activity, especially acts selectively on yeast-type fungi, with a minimal inhibitory concentration (MIC) in a range of 70-100 μg/ml. However, there has been no report at home and abroad on the use of baicalin in the treatment of poisoning caused by ricin.

Ricin is a highly toxic protein extracted from Castor seeds, belonging to the type II ribosome-inactivating protein family. Ricin has N-glycosidase activity and inhibits protein synthesis in mammalian cells, resulting in cell apoptosis and death. Therefore, ricin has been used as a biological warfare agent since the early 20th century, due to its high toxicity, easy extraction and high stability, and ricin can be easily exploited by terrorist organizations, posing a great threat to human health. US Centers for Disease Control and Prevention has referred to ricin as one of category B bioterrorism agents. So far, however, there has been no effective antidote against ricin in clinical practice. Consequently, research on small-molecule antagonists against the ricin activity is of great significance for rescuing ricin poisoning. The study of the present invention found that baicalin could inhibit cell death caused by ricin at a cellular level, and exhibited an obvious recovery effect on mouse ricin poisoning in vivo.

### SUMMARY OF THE INVENTION

Molecular structure of baicalin is as follows:

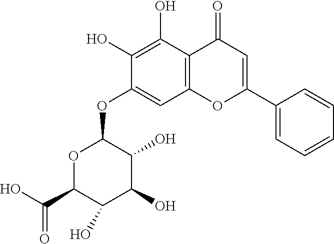 [SMILES: O=C(O)[C@H]1O[C@@H](Oc2cc3oc(-c4ccccc4)cc(=O)c3c(O)c2O)[C@H](O)[C@@H](O)[C@@H]1O]

The present invention has verified the therapeutic effects of baicalin on ricin infection by its protective effects on Hela cells and a mouse ricin poisoning model, and its mechanisms of action have been further clarified by a crystallography method.

### DETAILED DESCRIPTION OF THE EMBODIMENTS

### Example 1

### Protective Effects of Baicalin on Hela Cells

Cultured Hela cells were plated into a 96-well cell culture plate at a density of 15000 cells per well. After 24h incubation to allow complete adherence to surfaces, 100 pg of purified ricin was added to each well, and different concentrations of baicalin solution were added, then the cells were incubated in a CO₂ incubator. After incubation for 72 h, the culture medium supernatants were collected by centrifugation for detecting lactate dehydrogenase release amount. The protective effects of baicalin on the cells were evaluated by lactate dehydrogenase release. The result showed that baicalin significantly inhibited the cytotoxicity of Hela cells caused by ricin, in a dose-dependent manner. Survival rates of Hela cells after adding different concentrations of baicalin are shown in Table 1 below:

TABLE 1
Inhibitory effects of baicalin on the cytotoxicity
of Hela cells caused by ricin.

| Baicalin (μg/ml) | Survival rate (%) |
|---|---|
| 0 | 21.39 |
| 4 | 27.56 |
| 8 | 42.18 |
| 16 | 56.89 |
| 32 | 77.42 |

### Example 2

Analysis on Mechanisms of Baicalin against Ricin and Target Validation

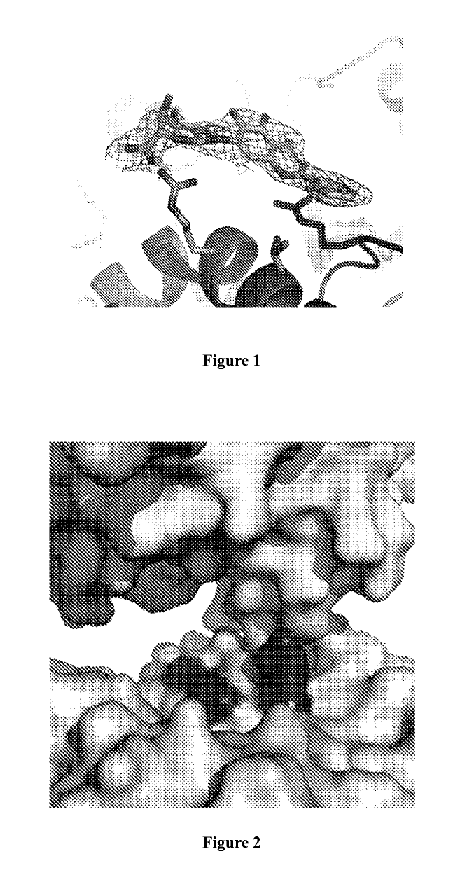

In order to clarify the mechanisms of baicalin against the ricin activity, the protein crystal of baicalin and ricin was obtained using the pendant drop method in the present study, and then the crystal structure of the complex of ricin with baicalin was obtained after crystal data were calculated by softwares, as shown in FIG. 1. By analysis it was found that baicalin could induce ricin to form a polymer after baicalin reacted with ricin, then active sites of ricin were blocked after polymerisation, resulting in the loss of most activity, and the binding sites of baicalin to ricin (FIG. 2) were Arg189, Thr190, Arg193, Tyr194, Arg235 and Arg258. It was also found by verification of further experiments that the major action sites of baicalin on ricin were Arg189, Thr190, Arg193, Tyr194 and Arg235.

### Example 3

### Study on Experimental Acology of Ricin Poisoning in Mice

3.1 Mouse Model of Ricin Poisoning

Male BALB/C mice, weighing 18-22 g, were anesthetized with ethyl ether, and were given purified ricin protein by intraperitoneal injection, then the mice were kept lying on their back until they regained consciousness. In this way, a mouse model of ricin poisoning was successfully established. For the survival rate experiment and pathology experiment, the mice were given 100 ng of purified ricin protein.

3.2 Protective Rate Test.

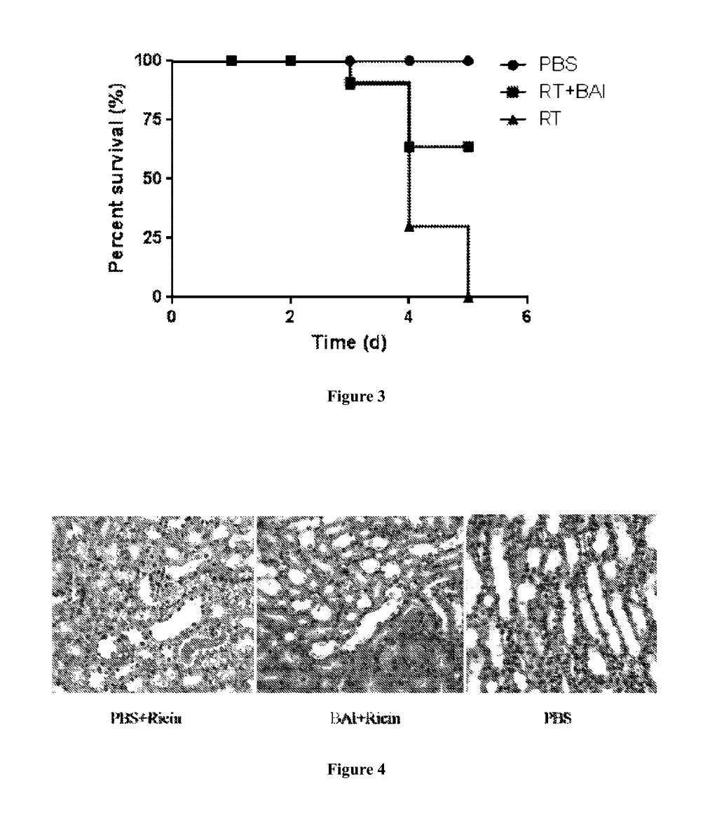

6 h after injection with ricin protein, mice in the drug administration group were given subcutaneous injection of 200 mg/kg baicalin, one dose every 6 h. Mice in the model control group were given 100 pl of sterilized PBS (20 in each group). Then mortality rates were statistically analyzed. The results showed that the survival rates of mice with ricin poisoning were significantly increased after baicalin treatment, as shown in FIG. 3.

3.3 Histopathological Experiment.

6 h after injection with ricin protein, mice in the drug administration group were given subcutaneous injection of 200 mg/kg baicalin, one dose every 6 h. Mice in the model control group were given 100 μl of sterilized PBS (10 mice in each group). 72 h after the infection, mice were euthanized under anesthesia and kidneys were enucleated for making pathological sections, then pathological changes were observed. The results showed that in mice of the model control group, renal hemorrhage appeared, large numbers of epithelial cells shed from renal tubules, epithelial cell casts were found, and renal glomeruli swelled. While in mice of the drug administration group, only a minor hemorrhage existed in the kidney tissue, and there was no significant difference compared with mice in normal group, as shown in FIG. 4.

1. A use of baicalin in preparation of a drug for treating and rescuing ricin poisoning.

2. The use according to claim 1, wherein the drugs are prepared into any pharmaceutically acceptable dosage form, comprising injections, capsules, tablets and powders for injection.

3. The use according to claim 1, wherein the ricin poisoning comprises types of poisoning induced by inhalation, ingestion and injection.
